(19) United States
(12) Patent Application Publication
(10) Pub. No.: US 2025/0017906 A1
(43) Pub. Date: Jan. 16, 2025

(54) COMPOUNDS FOR USE IN TREATING GASTRIC CANCER

(71) Applicant: BPGBio, Inc., Framingham, MA (US)

(72) Inventor: Vivek K. Vishnudas, Bedford, MA (US)

(21) Appl. No.: 18/711,570

(22) PCT Filed: Nov. 17, 2022
(86) PCT No.: PCT/US2022/050289

§ 371 Date: May 17, 2024

Related U.S. Application Data
(60) Provisional application No. 63/280,540, filed on Nov. 17, 2021.

(51) Int. Cl. A61K 31/4196; A61P 35/04

(52) U.S. Cl. CPC A61K 31/4196; A61P 35/04

(57) ABSTRACT

Provided are methods for treating gastric cancer using compounds and compositions comprising compounds having the formula (I): and pharmaceutically acceptable salts thereof.

### RELATED APPLICATIONS

This application claims the benefit of priority to U.S. Provisional Application No. 63/280,540, filed Nov. 17, 2021, the entire contents of which are incorporated herein by reference.

### BACKGROUND

The American Cancer Society estimates about 26,560 new cases of gastric cancer (also known as stomach cancer) this year with an estimated 11,180 deaths arising from this type of cancer. About 6 of every 10 people diagnosed with stomach cancer are 65 years of age or older. Gastric cancer is difficult to treat and exhibits poor survival with current therapies (five-year survival for all stages is 32% and is 5% for metastatic gastric cancer) demonstrating a clear unmet clinical need.

### SUMMARY

It has now been found that 2-(difluoromethoxy)-N-[[5-(2-methoxyphenyl)-1H-1,2,4-triazol-3-yl]methyl]benzamide, significantly decreases the growth of tumors in a human xenograft model for gastric cancer. See e.g., FIG. 1 where 2-(difluoromethoxy)-N-[[5-(2-methoxyphenyl)-1H-1,2,4-triazol-3-yl]methyl]benzamide demonstrated more than 70% HS746T tumor growth inhibition (TGI) at 150 mg/kg in nude mice.

Provided herein, therefore, are methods of treating gastric cancer using 2-(difluoromethoxy)-N-[[5-(2-methoxyphenyl)-1H-1,2,4-triazol-3-yl]methyl]benzamide, or a pharmaceutically acceptable salt thereof.

Also provided herein are methods of treating gastric cancer using a compound of Formula I:

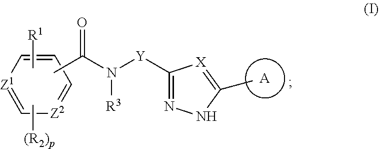

or a pharmaceutically acceptable salt or composition thereof, wherein A, R¹, R², R³, Z¹, Z² and p are as defined herein.

### BRIEF DESCRIPTION OF THE FIGURES

FIG. 1 shows the tumor growth inhibitory effects on HS746T tumors in nude mice over a 15-day course treatment with 2-(difluoromethoxy)-N-[[5-(2-methoxyphenyl)-1H-1,2,4-triazol-3-yl]methyl]benzamide.

FIG. 2 shows the level of cyclin B1(CCNB1) and phospho histone H3(pHH3) from HS746T xenograft tumors in a xenograft model treated with 2-(difluoromethoxy)-N-[[5-(2-methoxyphenyl)-1H-1,2,4-triazol-3-yl]methyl]benzamide.

FIG. 3 shows the tumor growth inhibitory effects on SNU5 tumors in nude mice over a 21-day course treatment with 2-(difluoromethoxy)-N-[[5-(2-methoxyphenyl)-1H-1,2,4-triazol-3-yl]methyl]benzamide.

FIG. 4 illustrates the dose dependent increase of cyclin B1 and pHis H3 in tumors from treatment with 2-(difluoromethoxy)-N-[[5-(2-methoxyphenyl)-1H-1,2,4-triazol-3-yl]methyl]benzamide.

### DETAILED DESCRIPTION

Provided is a method of treating gastric cancer in a subject comprising administering to the subject a therapeutically effective amount of 2-(difluoromethoxy)-N-[[5-(2-methoxyphenyl)-1H-1,2,4-triazol-3-yl]methyl]benzamide, or a pharmaceutically acceptable salt thereof.

2-(difluoromethoxy)-N-[[5-(2-methoxyphenyl)-1H-1,2,4-triazol-3-yl]methyl]benzamide has the chemical structure shown below and may be synthesized according to the procedures described for compound 126 in U.S. Pat. No. 11,091,447, the entire contents of which are incorporated herein by reference.

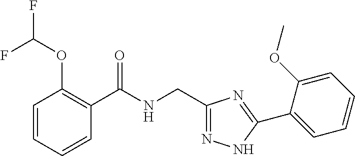 [SMILES: COc1ccccc1-c1nc(CNC(=O)c2ccccc2OC(F)F)n[nH]1]

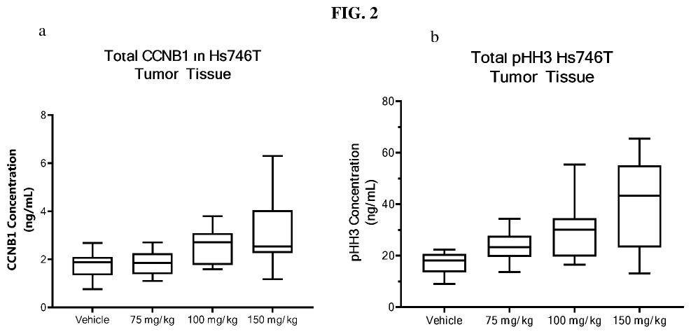

2-(difluoromethoxy)-N-[[5-(2-methoxyphenyl)-1H-1,2,4-triazol-3-yl]methyl]benzamide was previously shown to stabilize mono-ubiquitinated UBE2K in poly-ubiquitination assays, and is therefore considered to be, in one aspect, an effector of UBE2K. See e.g., FIG. 2 of U.S. Pat. No. 11,091,447. Recently, however, evidence suggests that 2-(difluoromethoxy)-N-[[5-(2-methoxyphenyl)-1H-1,2,4-triazol-3-yl]methyl]benzamide may also act as a modulator (e.g., an inhibitor) of microtubule assembly.

2-(difluoromethoxy)-N-[[5-(2-methoxyphenyl)-1H-1,2,4-triazol-3-yl]methyl]benzamide may exist in various tautomeric forms, each of which are expressly included as part of the invention.

Also provided is a method of treating gastric cancer in a subject comprising administering to the subject a pharmaceutical composition comprising a therapeutically effective amount of 2-(difluoromethoxy)-N-[[5-(2-methoxyphenyl)-1H-1,2,4-triazol-3-yl]methyl]benzamide, or a pharmaceutically acceptable salt thereof; and a pharmaceutically acceptable carrier.

Also provided is the use of 2-(difluoromethoxy)-N-[[5-(2-methoxyphenyl)-1H-1,2,4-triazol-3-yl]methyl]benzamide, or a pharmaceutically acceptable salt thereof, for the manufacture of a medicament for treating gastric cancer (e.g., in a subject).

Also provided is the use of 2-(difluoromethoxy)-N-[[5-(2-methoxyphenyl)-1H-1,2,4-triazol-3-yl]methyl]benzamide, or a pharmaceutically acceptable salt thereof, for treating gastric cancer (e.g., in a subject).

Also provided is a pharmaceutically acceptable composition comprising 2-(difluoromethoxy)-N-[[5-(2-methoxyphenyl)-1H-1,2,4-triazol-3-yl]methyl]benzamide, or a pharmaceutically acceptable salt thereof, for treating gastric cancer (e.g., in a subject).

Also provided is a method of treating gastric cancer in a subject comprising administering to the subject a therapeutically effective amount of a compound of Formula I:

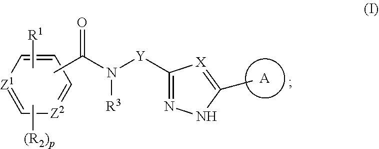

or a pharmaceutically acceptable salt thereof, wherein
Z¹ and Z² are each independently N or CH;
X is N or CH;
ring A is phenyl or a 5- to 9-membered heteroaryl, each of which are optionally substituted with 1 to 3 groups selected from R⁵;
Y is CH₂, —CHR^{a}, —CR^{a}R^{b}, or SO;
R^{a} and R^{b} are each independently halo, (C₁-C₆)alkyl, or halo(C₁-C₆)alkyl; or R^{a} and R^{b} together with the carbon atom they are bound for a 3- to 6-membered cycloalkyl or a 3- to 6-membered heterocyclyl, each of which are optionally substituted with 1 to 3 groups selected from halo, (C₁-C₆)alkyl, halo(C₁-C₆)alkyl, (C₁-C₆)alkoxy, halo(C₁-C₆)alkoxy, (C₁-C₆)alkylOH, (C₁-C₆)alkylO(C₁-C₆)alkyl, and OH;
R¹ is halo(C₁-C₆)alkyl, halo(C₁-C₆)alkoxy, or —NR^{c}R^{d}, wherein two available hydrogen atoms on said halo(C₁-C₆)alkyl and halo(C₁-C₆)alkoxy may be taken together to which the carbon atoms they are attached to form a 3- to 6-membered cycloalkyl optionally substituted with 1 to 3 groups selected from halo, (C₁-C₆)alkyl, halo(C₁-C₆)alkyl, (C₁-C₆)alkoxy, and halo(C₁-C₆)alkoxy;
R^{c} and R^{d} are each independently hydrogen (C₁-C₆)alkyl, halo(C₁-C₆)alkyl, (C₁-C₆)alkylO(C₁-C₆)alkyl, halo(C₁-C₆)alkylO(C₁-C₆)alkyl, (C₁-C₆)alkyl-O-halo(C₁-C₆)alkyl, halo(C₁-C₆)alkyl-O-halo(C₁-C₆)alkyl, or (C₁-C₆)alkylOH; or R^{c} and R^{d} together with the nitrogen atom they are bound form a 4- to 7-membered heterocyclyl optionally substituted with 1 to 3 groups selected from halo, (C₁-C₆)alkyl, halo(C₁-C₆)alkyl, (C₁-C₆)alkoxy, halo(C₁-C₆)alkoxy, and oxo;
R² is CN, halo, OH, (C₁-C₆)alkyl, halo(C₁-C₆)alkyl, (C₁-C₆)alkoxy, or halo(C₁-C₆)alkoxy; or R¹ and R², when on adjacent carbon atoms, are taken together with the carbon atoms to which they are attached to form a 5- or 6-membered oxygen containing heterocyclyl optionally substituted with 1 to 3 groups selected from halo, (C₁-C₆)alkyl, and halo(C₁-C₆)alkyl;
R³ is hydrogen, (C₁-C₆)alkyl, or halo(C₁-C₆)alkyl;
R⁴ is CN, halo, OH, (C₁-C₆)alkyl, halo(C₁-C₆)alkyl, (C₁-C₆)alkoxy, halo(C₁-C₆)alkoxy, —NH(C₁-C₆)alkyl, —N[(C₁-C₆)alkyl]2, or a 5- to 6-membered heterocyclyl; and
p is 0 or 1.

Also provided is a method of treating gastric cancer in a subject comprising administering to the subject a pharmaceutical composition comprising a therapeutically effective amount of a compound of Formula I, or a pharmaceutically acceptable salt thereof; and a pharmaceutically acceptable carrier.

Also provided is the use of a compound of Formula I, or a pharmaceutically acceptable salt thereof, for the manufacture of a medicament for treating gastric cancer (e.g., in a subject).

Also provided is the use of a compound of Formula I, or a pharmaceutically acceptable salt thereof, for treating gastric cancer (e.g., in a subject).

Also provided is a pharmaceutically acceptable composition comprising a compound of Formula I, or a pharmaceutically acceptable salt thereof, for treating gastric cancer (e.g., in a subject).

In one aspect, the compound of Formula I is of the Formula II or III:

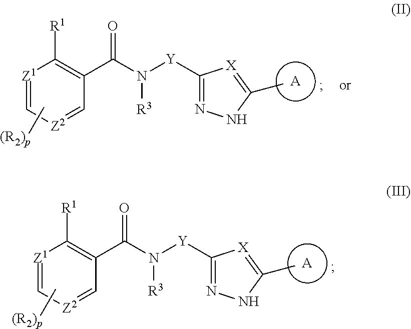

or a pharmaceutically acceptable salt thereof, wherein the remaining variables are as described above for Formula I.

In one aspect, the compound of Formula I is of the Formula IV:

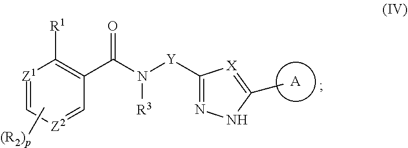

or a pharmaceutically acceptable salt thereof, wherein the remaining variables are as described above for Formula I.

In one aspect, R³ in the compound of Formula I, II, III, or IV, or a pharmaceutically acceptable salt thereof, is hydrogen, wherein the remaining variables are as described for Formula I.

In one aspect, Y in the compound of Formula I, II, III, or IV, or a pharmaceutically acceptable salt thereof, is CH₂, SO₂, or cyclopropyl, wherein the remaining variables are as described for Formula I or any of the above aspects. Alternatively, Y in the compound of Formula I, II, III, or IV, or a pharmaceutically acceptable salt thereof, is CH₂, wherein the remaining variables are as described for Formula I or any of the above aspects.

In one aspect, Z¹ is N and Z² is CH; Z¹ is CH and Z² is N; or Z¹ and Z² are each CH in the compound of Formula I, II, III, or IV, or a pharmaceutically acceptable salt thereof, wherein the remaining variables are as described for Formula I or any of the above aspects. Alternatively, Z¹ and Z² are each CH in the compound of Formula I, II, III, or IV, or a pharmaceutically acceptable salt thereof, wherein the remaining variables are as described for Formula I or any of the above aspects.

In one aspect, ring A in the compound of Formula I, II, III, or IV, or a pharmaceutically acceptable salt thereof, is phenyl or a 5- to 6-membered heteroaryl, each of which are optionally substituted with 1 to 3 groups selected from R⁵, wherein the remaining variables are as described for Formula I or any of the above aspects. Alternatively, ring A in the compound of Formula I, II, III, or IV, or a pharmaceutically acceptable salt thereof, is phenyl, pyridyl, furanyl, or pyrazolyl, each of which are optionally substituted with 1 to 3 groups selected from R⁵, wherein the remaining variables are as described for Formula I or any of the above aspects. In another alternative, ring A in the compound of Formula I, II, III, or IV, or a pharmaceutically acceptable salt thereof, is phenyl or furanyl, each of which are optionally substituted with 1 to 3 groups selected from R⁵, wherein the remaining variables are as described for Formula I or any of the above aspects. In another alternative, ring A in the compound of Formula I, II, III, or IV, or a pharmaceutically acceptable salt thereof, is phenyl optionally substituted with 1 to 3 groups selected from R⁵, wherein the remaining variables are as described for Formula I or any of the above aspects.

In one aspect, R¹ and R², in the compound of Formula I, II, III, or IV, or a pharmaceutically acceptable salt thereof, are on adjacent carbon atoms and are taken together with the carbon atoms they are attached to form a 5-membered oxygen containing heterocyclyl optionally substituted with 1 or 2 halo, wherein the remaining variables are as described for Formula I or any of the above aspects. Alternatively, R¹ and R², in the compound of Formula I, II, III, or IV, or a pharmaceutically acceptable salt thereof, are on adjacent carbon atoms and are taken together with the carbon atoms they are attached to form a dioxolanyl optionally substituted with 1 or 2 halo, wherein the remaining variables are as described for Formula I or any of the above aspects.

In one aspect, R¹ in the compound of Formula I, II, III, or IV, or a pharmaceutically acceptable salt thereof, is halo(C₁-C₄)alkyl, halo(C₁-C₄)alkoxy, or —NR^{c}R^{d}; and R^{c} is hydrogen and R^{d} is halo(C₁-C₄)alkyl; or R^{c} and R^{d} are taken together to form a 4- to 7-membered heterocyclyl optionally substituted with 1 to 3 groups selected from halo, (C₁-C₄)alkyl, and oxo, wherein the remaining variables are as described for Formula I or any of the above aspects. Alternatively, R¹ in the compound of Formula I, II, III, or IV, or a pharmaceutically acceptable salt thereof, is —OCF₃, —OCHF₂, —OCH₂CF₃, —CF₃, —CH₂CF₃, —CHF₂, piperidinyl, pyrrolidinyl, azapanyl, morpholinyl, thiomorpholinyl, piperazinyl, or azetidinyl and wherein each of said heterocyclic ring is optionally substituted with 1 to 3 groups selected from halo, (C₁-C₄)alkyl, and oxo, wherein the remaining variables are as described for Formula I or any of the above aspects.

In one aspect, R² in the compound of Formula I, II, III, or IV, or a pharmaceutically acceptable salt thereof, is CN, halo, (C₁-C₄)alkyl, halo(C₁-C₄)alkyl, or (C₁-C₄)alkoxy, wherein the remaining variables are as described for Formula I or any of the above aspects. Alternatively, R² in the compound of Formula I, II, III, or IV, or a pharmaceutically acceptable salt thereof, is CN or halo, wherein the remaining variables are as described for Formula I or any of the above aspects. In another alternative, R² in the compound of Formula I, II, III, or IV, or a pharmaceutically acceptable salt thereof, is fluoro, wherein the remaining variables are as described for Formula I or any of the above aspects.

In one aspect, p in the compound of Formula I, II, III, or IV, or a pharmaceutically acceptable salt thereof, is 0, wherein the remaining variables are as described for Formula I or any of the above aspects.

In one aspect, R⁵ in the compound of Formula I, II, III, or IV, or a pharmaceutically acceptable salt thereof, is halo, (C₁-C₄)alkyl, halo(C₁-C₄)alkyl, (C₁-C₄)alkoxy, —N[(C₁-C₄)alkyl]₂, or a 6-membered heterocyclyl, wherein the remaining variables are as described for Formula I or any of the above aspects. Alternatively, R⁵ in the compound of Formula I, II, III, or IV, or a pharmaceutically acceptable salt thereof, is F, Br, Cl, —OCH₃, —OCH₂CH₃, OH, —O(CH₂)₂CH₃, —NMe₂, —CH(CH₃)₂, —C(CH₃)₃, —OCH(CH₃)₂, morpholinyl, —CH₃, or —CF₃, wherein the remaining variables are as described for Formula I or any of the above aspects.

In some aspects, the compound of Formula I is selected from any one of the following or a pharmaceutically acceptable salt thereof:

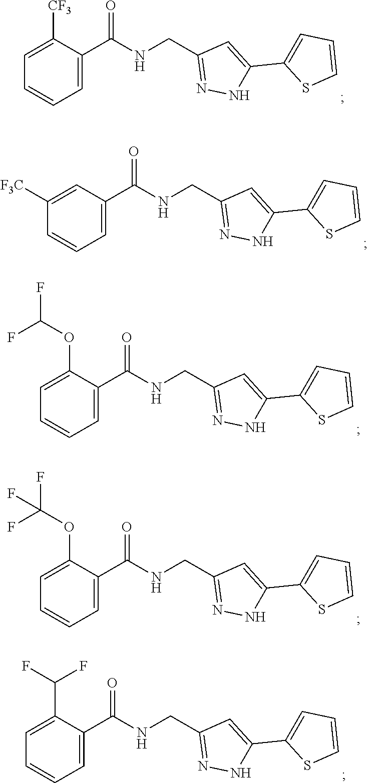

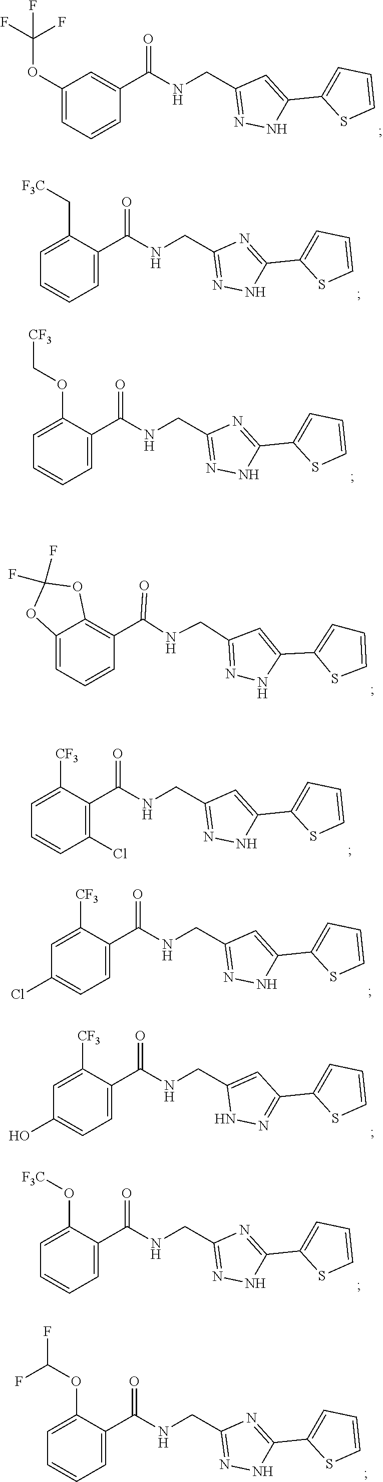

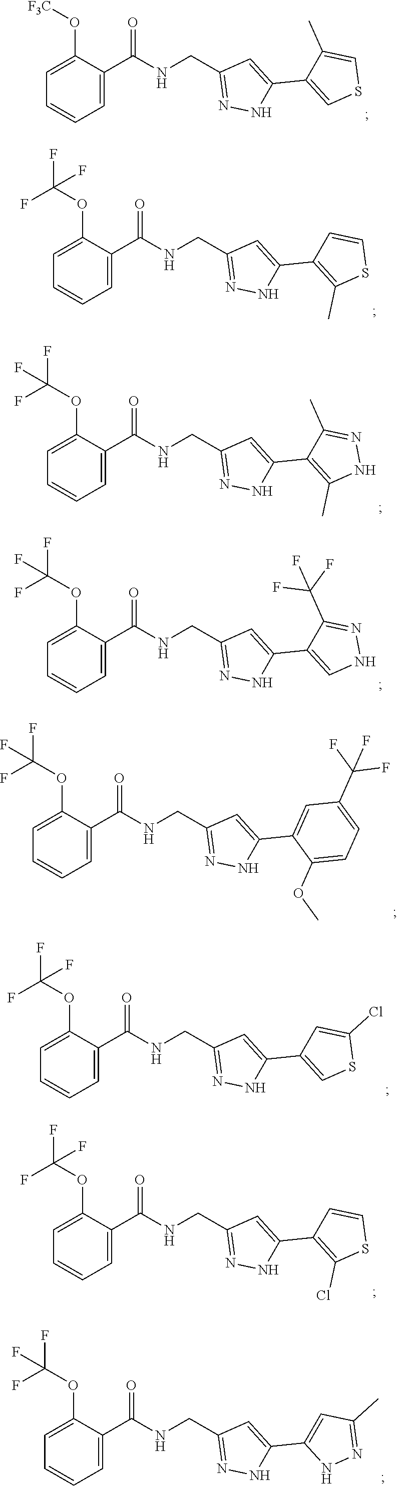

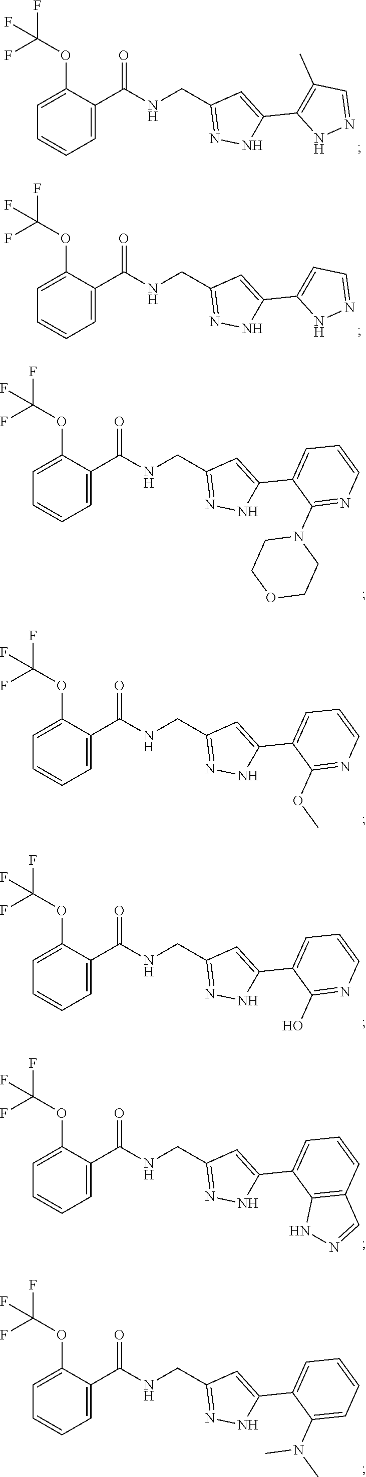

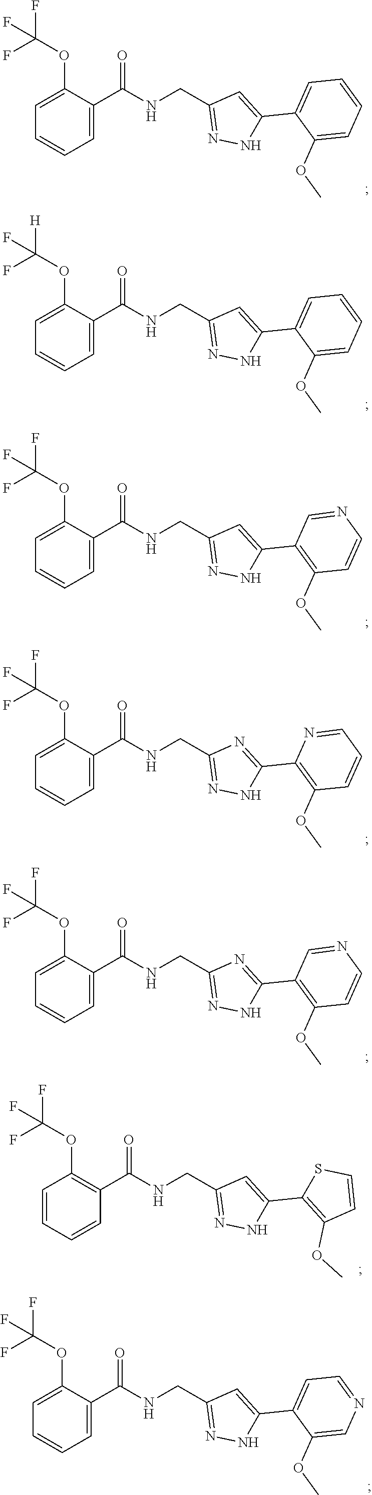

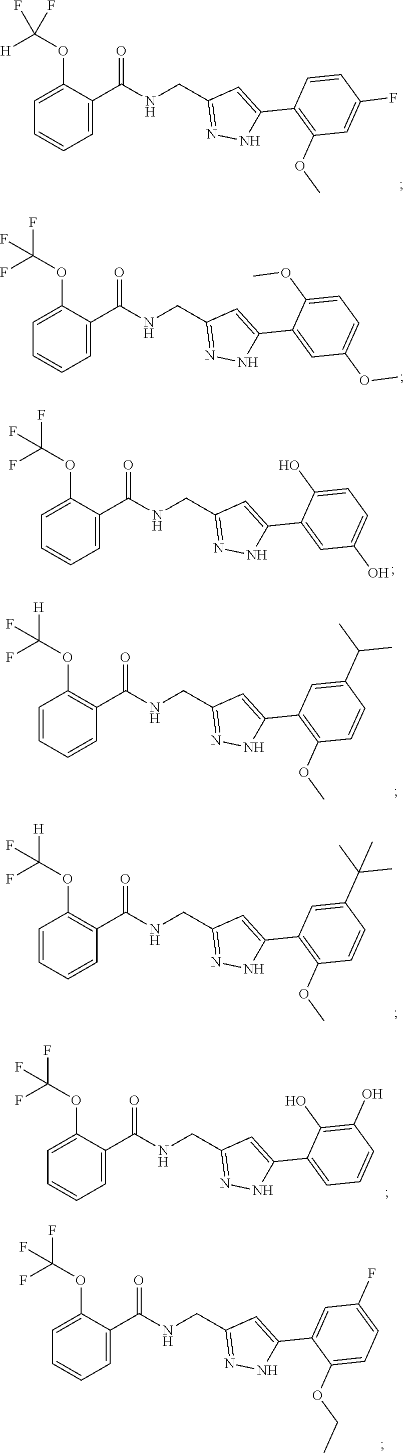

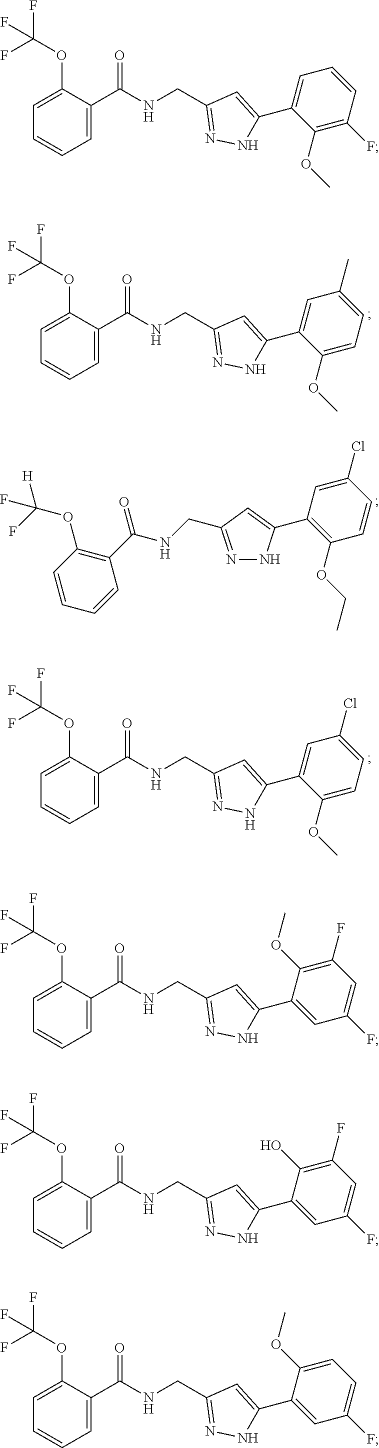

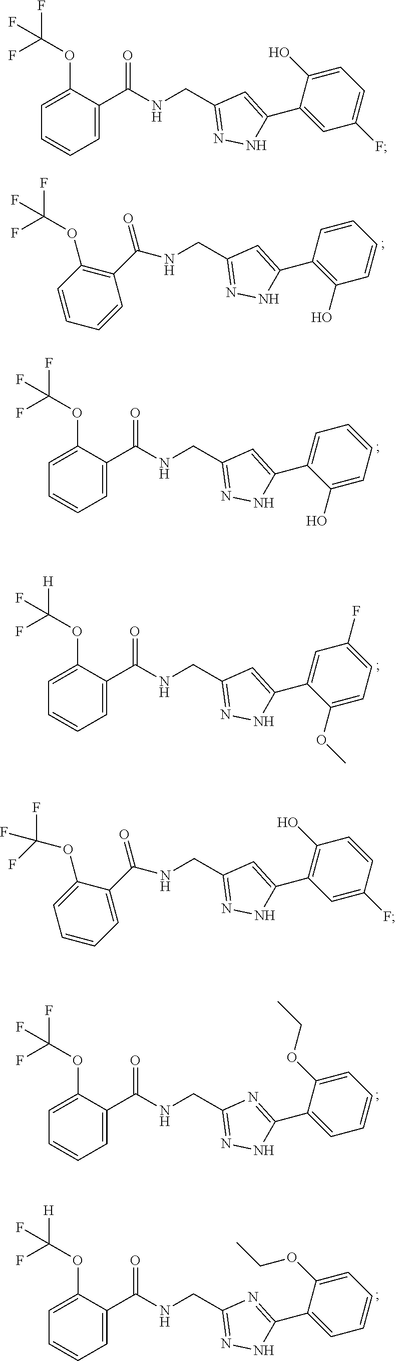

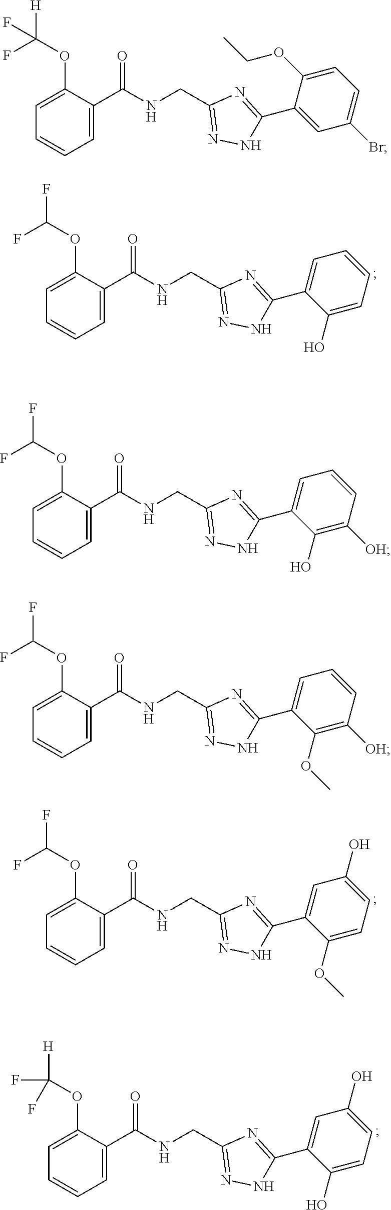

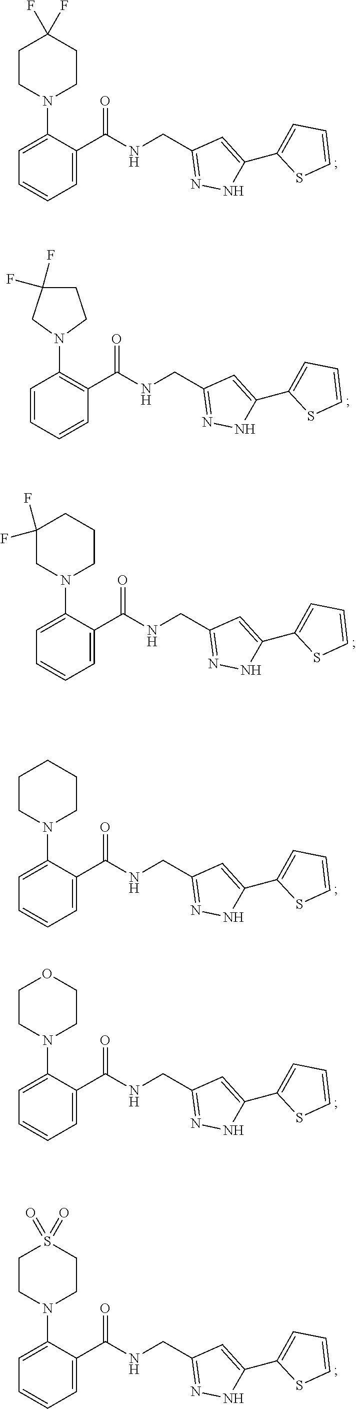

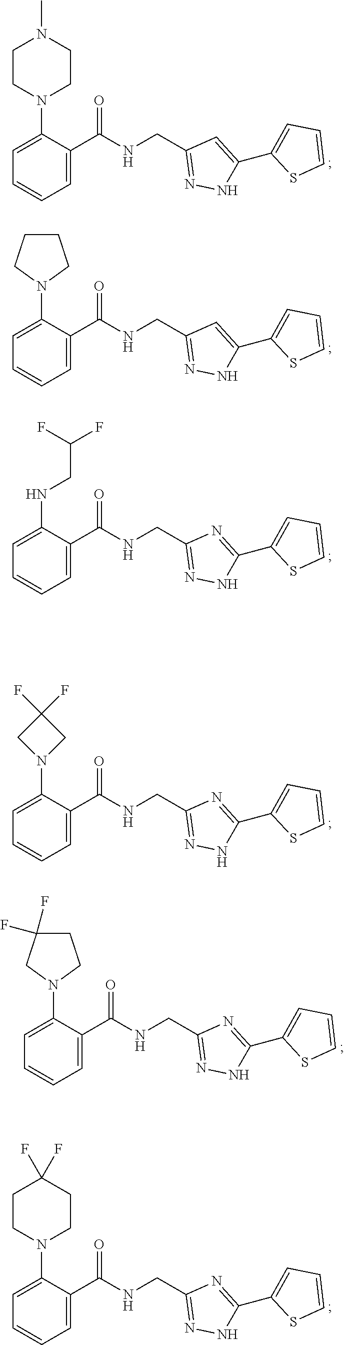

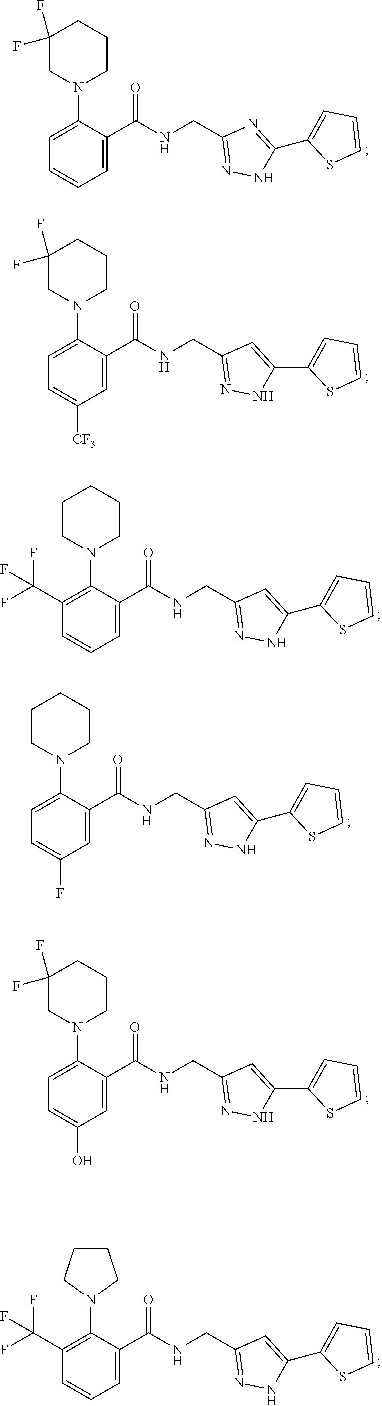

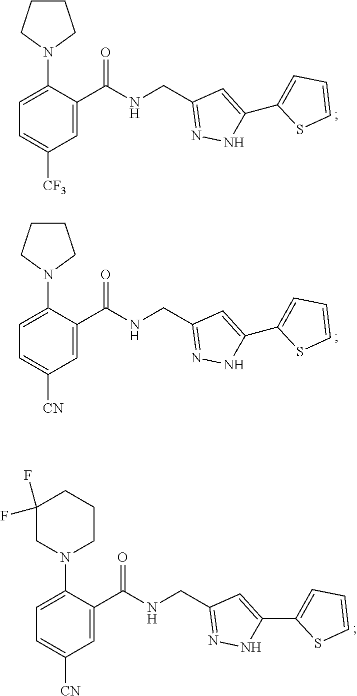

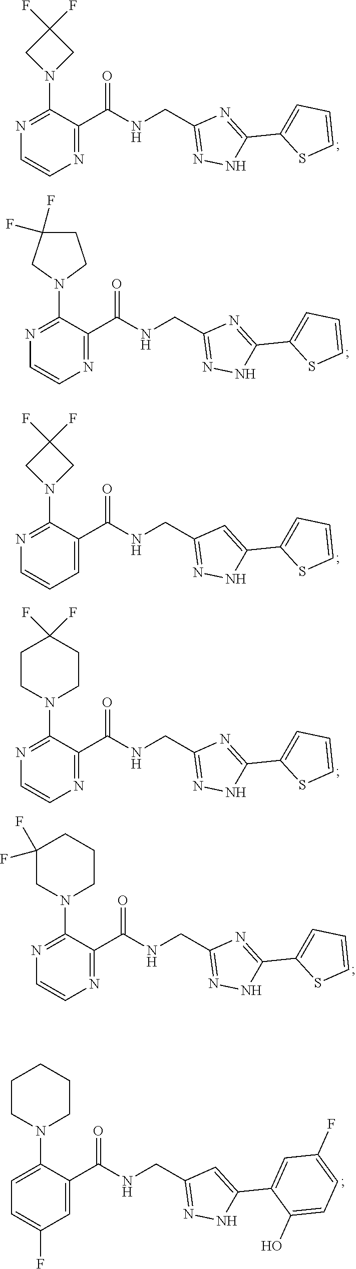

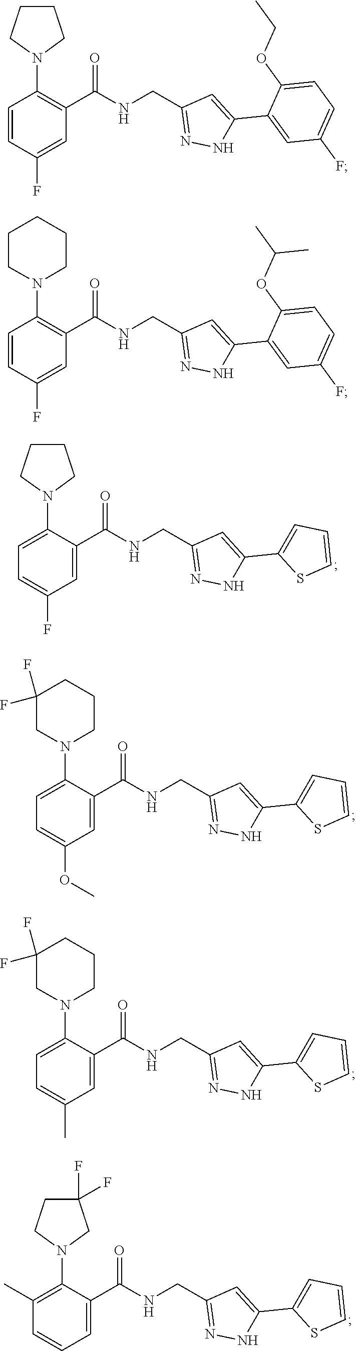

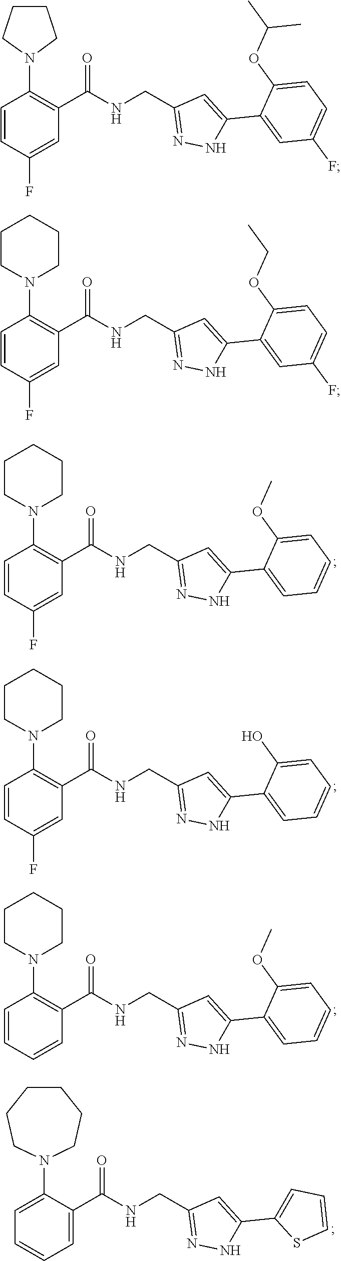

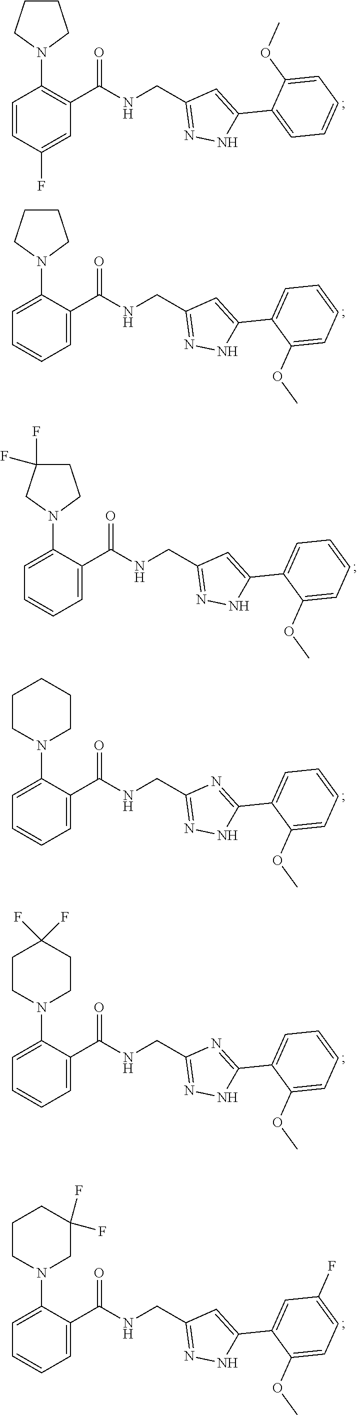

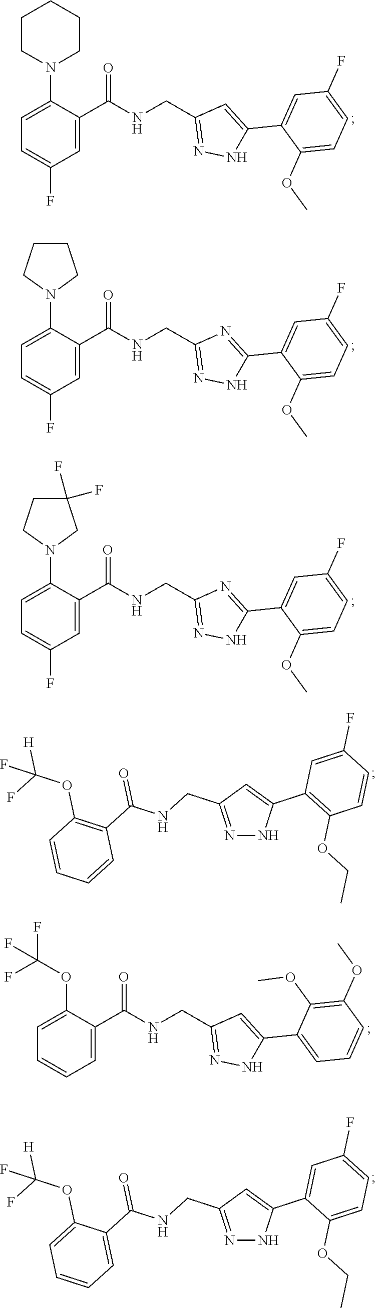

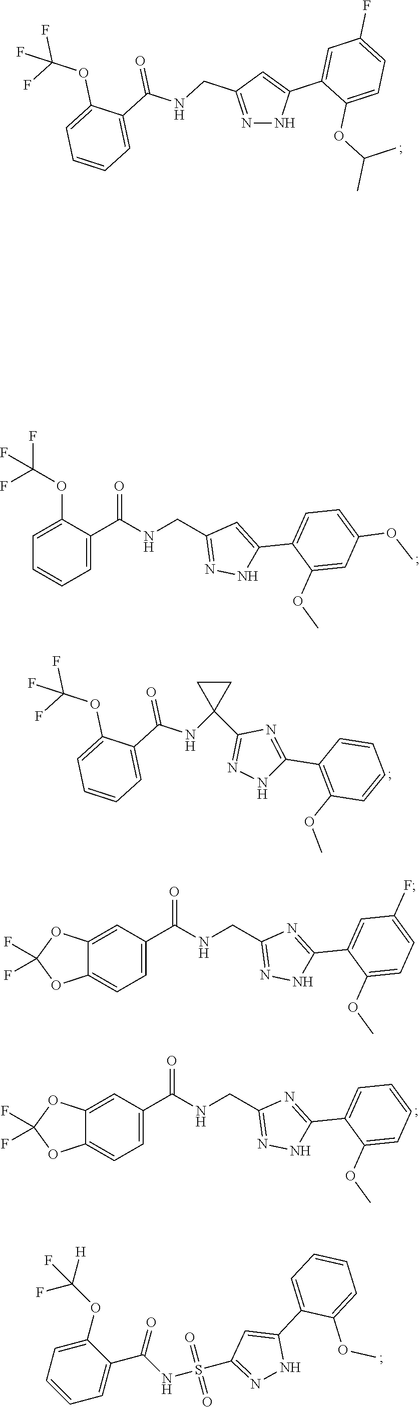 [SMILES: CC(C)Oc1ccc(F)cc1-c1cc(CNC(=O)c2ccccc2OC(F)(F)F)n[nH]1 ; COc1ccc(-c2cc(CNC(=O)c3ccccc3OC(F)(F)F)n[nH]2)c(OC)c1 ; COc1ccccc1-c1nc(C2(NC(=O)c3ccccc3OC(F)(F)F)CC2)n[nH]1 ; COc1ccc(F)cc1-c1nc(CNC(=O)c2ccc3c(c2)OC(F)(F)O3)n[nH]1 ; COc1ccccc1-c1nc(CNC(=O)c2ccc3c(c2)OC(F)(F)O3)n[nH]1 ; COc1ccccc1-c1cc(S(=O)(=O)NC(=O)c2ccccc2OC(F)F)n[nH]1]

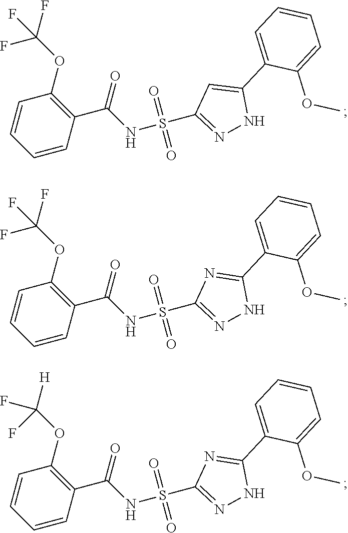

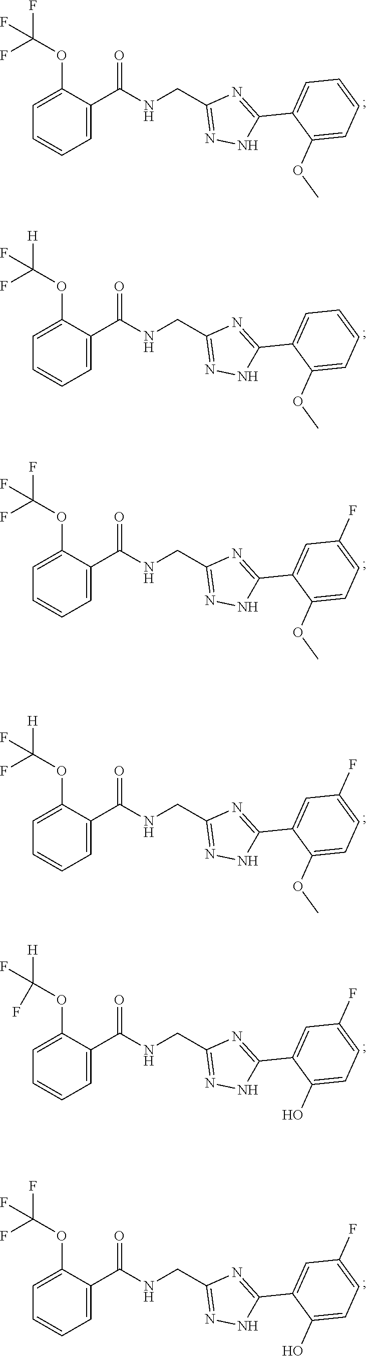 [SMILES: COc1ccccc1-c1nc(CNC(=O)c2ccccc2OC(F)(F)F)n[nH]1 ; COc1ccccc1-c1nc(CNC(=O)c2ccccc2OC(F)F)n[nH]1 ; COc1ccc(F)cc1-c1nc(CNC(=O)c2ccccc2OC(F)(F)F)n[nH]1 ; COc1ccc(F)cc1-c1nc(CNC(=O)c2ccccc2OC(F)F)n[nH]1 ; O=C(NCc1n[nH]c(-c2cc(F)ccc2O)n1)c1ccccc1OC(F)F ; O=C(NCc1n[nH]c(-c2cc(F)ccc2O)n1)c1ccccc1OC(F)(F)F]

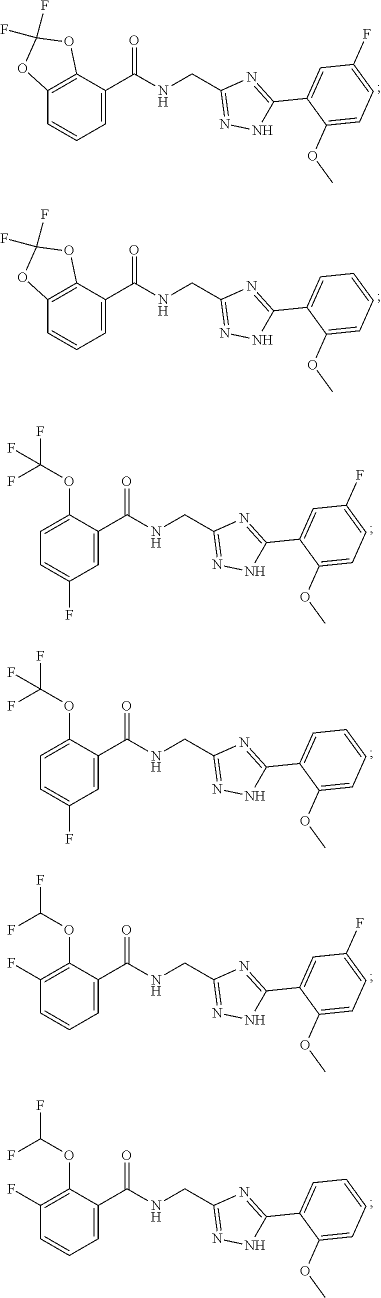 [SMILES: COc1ccc(F)cc1-c1nc(CNC(=O)c2cccc3c2OC(F)(F)O3)n[nH]1 ; COc1ccccc1-c1nc(CNC(=O)c2cccc3c2OC(F)(F)O3)n[nH]1 ; COc1ccc(F)cc1-c1nc(CNC(=O)c2cc(F)ccc2OC(F)(F)F)n[nH]1 ; COc1ccccc1-c1nc(CNC(=O)c2cc(F)ccc2OC(F)(F)F)n[nH]1 ; COc1ccc(F)cc1-c1nc(CNC(=O)c2cccc(F)c2OC(F)F)n[nH]1 ; COc1ccccc1-c1nc(CNC(=O)c2cccc(F)c2OC(F)F)n[nH]1]

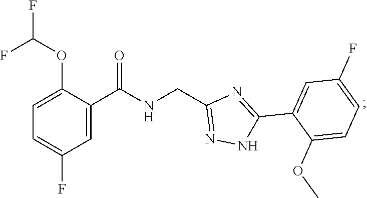 [SMILES: COc1ccc(F)cc1-c1nc(CNC(=O)c2cc(F)ccc2OC(F)F)n[nH]1]

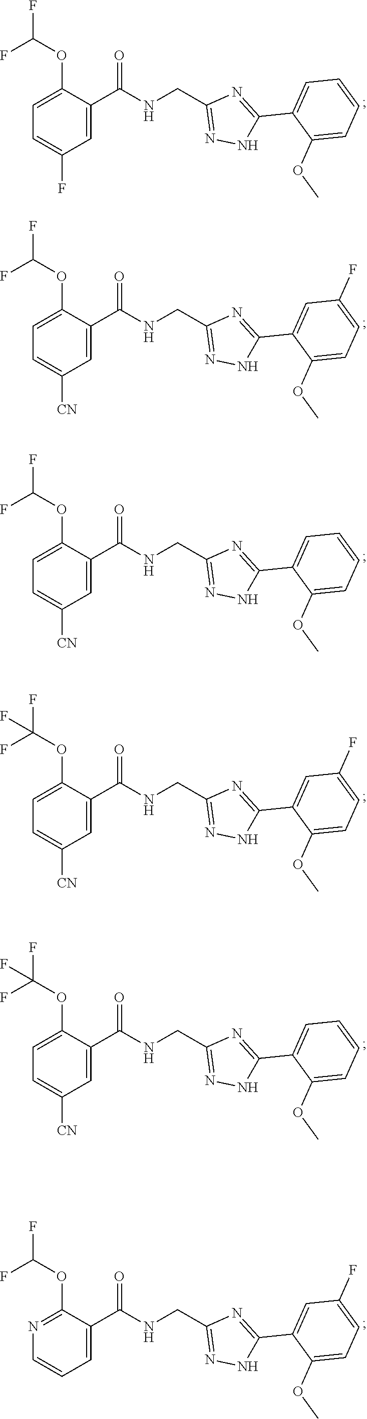 [SMILES: COc1ccccc1-c1nc(CNC(=O)c2cc(F)ccc2OC(F)F)n[nH]1 ; COc1ccc(F)cc1-c1nc(CNC(=O)c2cc(C#N)ccc2OC(F)F)n[nH]1 ; COc1ccccc1-c1nc(CNC(=O)c2cc(C#N)ccc2OC(F)F)n[nH]1 ; COc1ccc(F)cc1-c1nc(CNC(=O)c2cc(C#N)ccc2OC(F)(F)F)n[nH]1 ; COc1ccccc1-c1nc(CNC(=O)c2cc(C#N)ccc2OC(F)(F)F)n[nH]1 ; COc1ccc(F)cc1-c1nc(CNC(=O)c2cccnc2OC(F)F)n[nH]1]

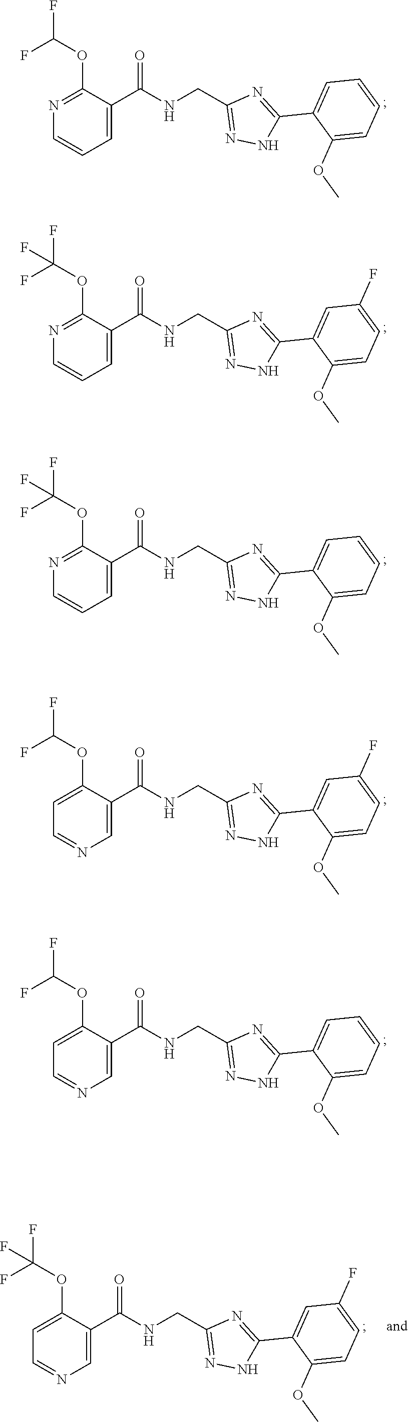 [SMILES: COc1ccccc1-c1nc(CNC(=O)c2cccnc2OC(F)F)n[nH]1 ; COc1ccc(F)cc1-c1nc(CNC(=O)c2cccnc2OC(F)(F)F)n[nH]1 ; COc1ccccc1-c1nc(CNC(=O)c2cccnc2OC(F)(F)F)n[nH]1 ; COc1ccc(F)cc1-c1nc(CNC(=O)c2cnccc2OC(F)F)n[nH]1 ; COc1ccccc1-c1nc(CNC(=O)c2cnccc2OC(F)F)n[nH]1 ; COc1ccc(F)cc1-c1nc(CNC(=O)c2cnccc2OC(F)(F)F)n[nH]1]

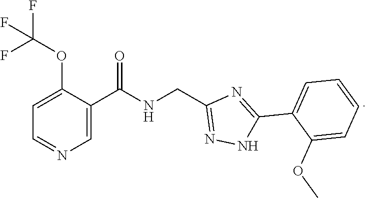 [SMILES: COc1ccccc1-c1nc(CNC(=O)c2cnccc2OC(F)(F)F)n[nH]1]

The foregoing compounds can be prepared following the procedures described in U.S. Pat. No. 11,091,447.

In one aspect, the gastric cancer treated by the present methods is metastatic.

When used in connection to describe a chemical group that may have multiple points of attachment, a hyphen (-) designates the point of attachment of that group to the variable to which is defined. For example, —NH(C₁-C₆)alkyl means that the point of attachment for this group is on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, means saturated straight-chain or branched monovalent hydrocarbon radical. Unless otherwise specified, an alkyl group typically has 1-4 carbon atoms, i.e., (C₁-C₄)alkyl.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., but are not limited to —OCHCF₂ or —OCF₃.

“Oxo” refers to the divalent function group ═O, i.e., an oxygen atom connected to another atom (typically carbon or sulfur) by a double bond.

The term “heteroaryl” refers to an aromatic ring of the specified size (e.g., 5-, 6-, 7-, 8-, or 9-membered ring) containing 1 to 4 heteroatoms independently selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. Bi-cyclic heteroaryl include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, pyrazolopyridinyl, thienopyridinyl, thienopyrimidinyl, indolizinyl, etc. When specified, optional substituents on a heteroaryl group may be present on any substitutable position.

The term “heterocyclyl” refers to a saturated or partially unsaturated monocyclic ring of the specified size (e.g., 3-, 4-, 5-, 6-, or 7-membered ring) containing 1 to 4 heteroatoms independently selected from N, O, and S. A heterocyclyl ring can be attached to its pendant group at any heteroatom or carbon atom that results in a stable structure. Examples of such saturated or partially unsaturated heterocyclic radicals include, without limitation, oxiranyl, thiiranyl, aziridinyl, tetrahydrofuranyl, tetrahydrothienyl, terahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. When specified, optional substituents on a heterocyclyl group may be present on any substitutable position and, include, e.g., the position at which the heterocyclyl is attached.

The term “cycloalkyl” refers to a monocyclic hydrocarbon of the specified size (e.g., 3-, 4-, 5-, 6-, or 7-membered ring). Cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. When specified, optional substituents on a cycloalkyl group may be present on any substitutable position and, include, e.g., the position at which the cycloalkyl is attached.

The disclosed compounds exist in various tautomeric forms and are part of the present disclosure. The term “tautomers” or “tautomeric” refers to two or more interconvertible compounds/substituents resulting from at least one formal migration of a hydrogen atom and at least one change in valency. Exemplary tautomerizations include e.g., the following:

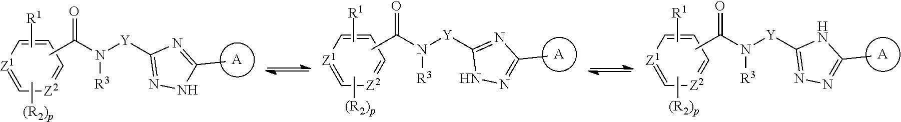

All such isomeric forms of such compounds are expressly included. Thus, when a compound herein is represented by a structural formula or designated by a chemical name herein, all other tautomeric forms which may exist for the compound are encompassed by the structural formula. This includes compounds of the Formula I where X is N or C.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The terms “subject” and “patient” may be used interchangeably, and refer to a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the terms “treatment,” “treat,” and “treating” refer to reversing, alleviating, delaying the onset of, or inhibiting the progress of gastric cancer, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “effective amount” or “therapeutically effective amount” refers to an amount of 2-(difluoromethoxy)-N-[[5-(2-methoxyphenyl)-1H-1,2,4-triazol-3-yl]methyl]benzamide that will elicit a biological or medical response of a subject e.g., a dosage of between 0.01-100 mg/kg body weight/day.

Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated.

### Exemplification

### Gastric Carcinoma Xenograft Model

Hs 746T cell line is a gastric carcinoma epithelial cell type from stomach that were isolated from the metastatic site of left leg muscle. Hs 746T cell lines were implanted subcutaneously in nude mice. Tumors were allowed to grow. Once the average tumor volume reached the volume of 120 mm3, animals were randomized into four groups of vehicle control, 75 mg/kg, 100 mg/kg, and 150 mg/kg 2-(difluoromethoxy)-N-[[5-(2-methoxyphenyl)-1H-1,2,4-triazol-3-yl]methyl]benzamide with 10 animals per group. Methocel E3 Premium LV with 2% labrasol in Milli-Q water was used for vehicle control. 2-(difluoromethoxy)-N-[[5-(2-methoxyphenyl)-1H-1,2,4-triazol-3-yl]methyl]benzamide was prepared prior to each dosing in 2% labrasol dissolved in Milli-Q water. 2-(difluoromethoxy)-N-[[5-(2-methoxyphenyl)-1H-1,2,4-triazol-3-yl]methyl]benzamide was administered by oral gavage two times a day (BID). In this study there were 10 animals per each group. All animals survived throughout the 15 days of the study. The mean tumor volume of each group over time and the growth of tumor in each individual animal is plotted below. For each individual animal of vehicle group TGI % was calculated based on the tumor growth inhibition of individual tumor at each given day of the study compared to the average of the group for that given day of the study. The P value was calculated for each group that was treated with 2-(difluoromethoxy)-N-[[5-(2-methoxyphenyl)-1H-1,2,4-triazol-3-yl]methyl]benzamide for each given day of the study by comparing the TGI of that day to the TGI of the vehicle group. This P value is different than what is provided in the CRO report which is the P value related to tumor volume not TGI %. P value was calculated by excel program using two-tailed distribution and two-sample equal variance (homoscedastic). ns P>0.05; * P<0.05; ** P<0.01; *** P<0.001; **** P<0.0001.

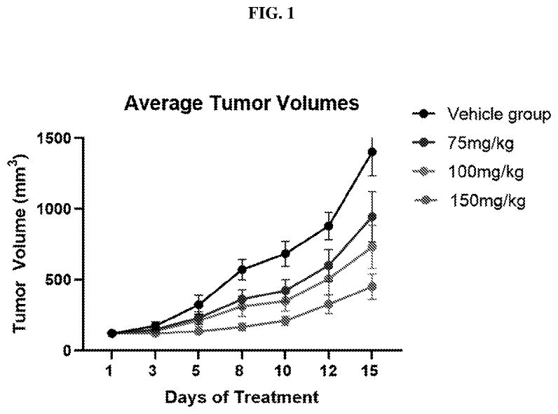

Statistical analysis using a mixed effect linear model of the tumor volumes on the last day of study demonstrated a tumor growth inhibition of 60.3%, 71.2% and 85.3% at the three doses, respectively, without any significant effect on body weight. See FIG. 1 and Table 1 below. These results are well correlated with the dose-dependent increase of cyclinB1 and phosphor histone H3 in response 2-(difluoromethoxy)-N-[[5-(2-methoxyphenyl)-1H-1,2,4-triazol-3-yl]methyl]benzamide in both tumor tissues. See FIG. 2.

TABLE 1
Effect of oral administration of 2-(difluoromethoxy)-N-[[5-
(2-methoxyphenyl)-1H-1,2,4-triazol-3-yl]methyl]benzamide
on Tumor Growth Inhibition (TGI) in a HS746T xenograft model
% Tumor Growth Inhibition (% TGI) on days

Days of study

| Dosage | 3 | 5 | 8 | 10 | 12 | 15 |
|---|---|---|---|---|---|---|
| 75 mg/kg | 47.2 | 45.9 | 46.3 | 46.3 | 36.8 | 35.7 |
| 100 mg/kg | 65.8 | 56.2 | 57.7 | 59.4 | 49.3 | 52.4 |
| 150 mg/kg | 98.3 | 93.1 | 89.8 | 84.2 | 72.9 | 74.3 |

SNU-5 is derived from ascites of a patient with poorly differentiated carcinoma of the stomach. The patient had previously received chemotherapy including 5-fluorouracil, doxorubicin and mitomycin-C. This cell line is derived from metastatic site, ascites.

SNU-5 cell lines were implanted subcutaneously in nude mice. Tumors were allowed to grow. Once the average tumor volume reached the volume of 122 mm3, animals were randomized into three groups of vehicle control, 75 mg/kg and 150 mg/kg 2-(difluoromethoxy)-N-[[5-(2-methoxyphenyl)-1H-1,2,4-triazol-3-yl]methyl]benzamide with 12 animals per group. Methocel E3 Premium LV in Milli-Q water was used for vehicle control. A solid dispersion of 2-(difluoromethoxy)-N-[[5-(2-methoxyphenyl)-1H-1,2,4-triazol-3-yl]methyl]benzamide was prepared prior to each dosing in Milli-Q water. 2-(difluoromethoxy)-N-[[5-(2-methoxyphenyl)-1H-1,2,4-triazol-3-yl]methyl]benzamide was administered by oral gavage two times a day (BID). In this study there were 12 animals per each group. All animals, except two animals from Group 2 (75 mg/kg), survived throughout the 23 days of the study. Animals that left the study prior to day 23 were eliminated from all calculations. The percentage of tumor growth inhibition (TGI %) was calculated for each individual tumor. The mean tumor volume of each group over time and the growth of tumor in each individual animal is plotted below. For each individual animal of vehicle group TGI % was calculated based on the tumor growth inhibition of individual tumor at each given day of the study compared to the average of the group for that given day of the study. The P value was calculated for each group that was treated with 2-(difluoromethoxy)-N-[[5-(2-methoxyphenyl)-1H-1,2,4-triazol-3-yl]methyl]benzamide for each given day of the study by comparing the TGI of that day to the TGI of the vehicle group. This P value is different than what is provided in the CRO report which is the P value related to tumor volume not TGI %. P value was calculated by excel program using two-tailed distribution and two-sample equal variance (homoscedastic). ns P>0.05; * P<0.05; ** P<0.01; *** P<0.001; **** P<0.0001.

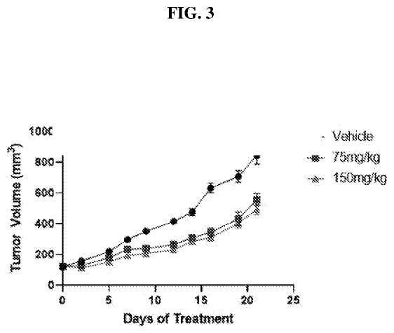

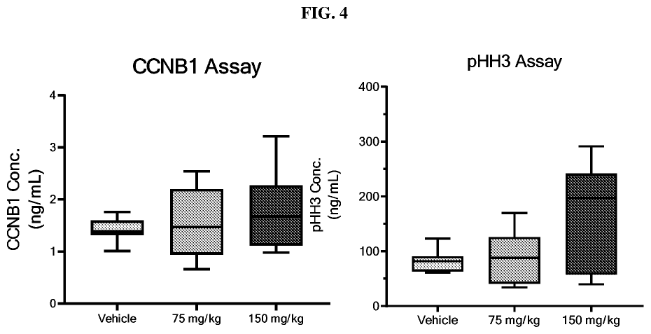

Results showed tumor growth inhibition of 64% at 150 mg/kg, without any significant effect on body weight. See FIG. 3 and Table 2 below. These results are well correlated with the dose-dependent increase of cyclin B1 and phosphor histone H3 in response to 2-(difluoromethoxy)-N-[[5-(2-methoxyphenyl)-1H-1,2,4-triazol-3-yl]methyl]benzamide in tumor tissues. See FIG. 4.

TABLE 2
% Tumor Growth Inhibition (% TGI) on days

| | Days of study | | | | | | | | |
|---|---|---|---|---|---|---|---|---|---|
| Dosage | 2 | 5 | 7 | 9 | 12 | 14 | 16 | 19 | 21 |
| 75 mg/kg | 79 | 41 | 36 | 49 | 51 | 47 | 56 | 47 | 40 |
| 150 mg/kg | 129 | 70 | 58 | 64 | 63 | 54 | 64 | 52 | 49 |

Unless otherwise defined, all technical and scientific terms used herein are accorded the meaning commonly known to one with ordinary skill in the art.

1. A method of treating gastric cancer in a subject in need thereof, comprising administering to the subject a therapeutically effective amount of 2-(difluoromethoxy)-N-[[5-(2-methoxyphenyl)-1H-1,2,4-triazol-3-yl]methyl]benzamide, or a pharmaceutically acceptable salt thereof.

2. The method of claim 1, wherein the gastric cancer is metastatic.
